(19) United States
(12) Patent Application Publication
(10) Pub. No.: US 2025/0057822 A1
(43) Pub. Date: Feb. 20, 2025

(54) PSYCHOPLASTOGENS FOR TREATING HEARING-RELATED DISORDERS

(71) Applicants: THE REGENTS OF THE UNIVERSITY OF CALIFORNIA, Oakland, CA (US); SALK INSTITUTE FOR BIOLOGICAL STUDIES, La Jolla, CA (US)

(72) Inventors: David E. OLSON, Oakland, CA (US); Uri MANOR, La Jolla, CA (US)

(21) Appl. No.: 18/720,410

(22) PCT Filed: Dec. 19, 2022
(86) PCT No.: PCT/US2022/081927

§ 371 Date: Jun. 14, 2024

Related U.S. Application Data
(60) Provisional application No. 63/291,003, filed on Dec. 17, 2021.

(51) Int. Cl. A61K 31/4375; A61K 31/4045; A61P 27/16

(52) U.S. Cl. CPC A61K 31/4375; A61K 31/4045; A61P 27/16

(57) ABSTRACT

Provided herein are psychoplastogens which can be useful for treating hearing loss.

### CROSS-REFERENCES TO RELATED APPLICATIONS

This application claims priority to U.S. Provisional Application No. 63/291,003, filed Dec. 17, 2021, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. R01GM128997 awarded by the National Institutes of Health. The Government has certain rights in this invention.

### BACKGROUND

Psychoplastogens are 5-HT2 agonists, which induce the formation of new synapses (“synaptogenesis”) and new neurites (“neuritogenesis”) in neurons expressing 5-HT2 receptors. Hearing loss is a major public issue that affects a large proportion of the population. In addition to psychological effects of hearing loss, hearing loss is also associated with a higher risk of other disorders, most notably dementia. Hearing loss is caused by the loss of synapses and neurites in the cochlea, which can result from noise or chemical exposure or due to the normal aging process. This invention describes the utilization of “psychoplastogens” to induce synaptogenesis and neuritogenesis in the cochlea. Since cochlear spiral ganglion neurons express 5-HT2 receptors, psychoplastogens can induce neuritogenesis/synaptogenesis in the cochlea.

The utilization of psychoplastogens to induce synaptogenesis in the ear has significant advantages over other current candidates such as BDNF or Trk agonists. While 5-HT2 agonists operate on the exact same pathways as BDNF and Trk agonists, they are much more specific, which can avoid off-target effects, whereas BDNF and Trk agonists will activate nearly all neurons in the nervous system, which can have undesirable effects. Furthermore, BDNF does not cross the blood brain barrier, whereas psychoplastogens readily do. The present invention meets this, and other, needs.

### BRIEF SUMMARY OF THE INVENTION

In one embodiment, the present invention provides a use of psychoplastogens for treating hearing loss.

### BRIEF DESCRIPTION OF THE DRAWINGS

FIG. 1 shows the effect of the psychoplastogen N,N-diethyl-8-methyl-7a,8,9,10-tetrahydro-7H-indolo [7,1-fg][1,7]naphthyridine-10-carboxamide on the synaptic density in the ear of a mouse.

FIG. 2A to 2D shows auditory brainstem response (ABR) wave I amplitudes. The ABR amplitudes were analyzed at baseline (5-6 days pre-treatment) and 24 h post-treatment of vehicle (FIG. 2A and FIG. 2B) or 1 mg/kg of the psychoplastogen psilocin (FIG. 2C and FIG. 2D) at 8 and 16 kHz. Data are presented as mean±SEM. Psilocin treatment after 24 h significantly increased wave I amplitude from baseline at 8 and 16 kHz (p<0.01, 2-way ANOVA followed by Sidak's multiple comparison test), but not vehicle control. N=3 8-week old C57/B6 female mice per condition.

FIG. 3 shows inner hair cell ribbon synapse density 24 h after psilocin treatment. C57/B6 female mice, aged 8-weeks-old were treated with psilocin (1 mg/kg) (n=3) and Vehicle (0.9% saline) (n=2). Psilocin treatment after 24 h significantly increased ribbon synapses in the 16 kHz region (Vehicle: 16±3.826, psilocin: 23.27±3.826, P=0.0169, 2-way ANOVA followed by Sidak's multiple comparison test). Synapse density increase was not significant in the 8 kHz region (Vehicle: 16.50±3.826, psilocin: 17.79±3.826, P=0.5554). For each animal, 17-22 inner hair cells were quantified per frequency (8 kHz and 16 kHz regions of the organ of Corti). Data are presented as mean±SEM.*p<0.05.

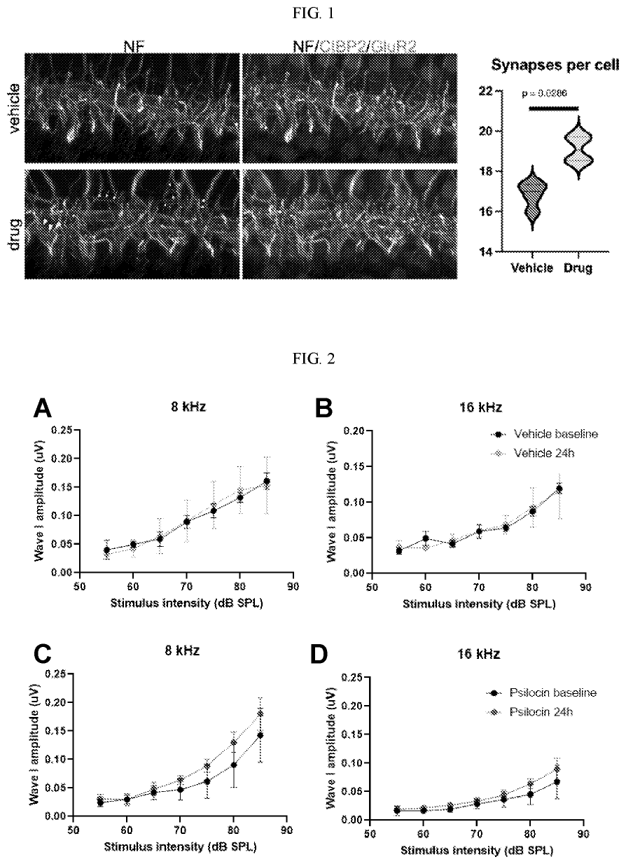

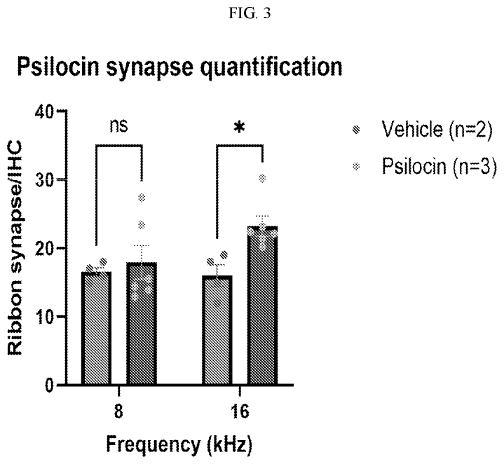

### DETAILED DESCRIPTION OF THE INVENTION

### I. General

Provided herein are tetracyclic ergoline analogs of heterocyclic compounds and tryptamine analogs such as psilocin. The compounds of the present invention are useful for treatment of diseases, such as hearing loss.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Disclosures provided herein of an “alkyl” are intended to include independent recitations of a saturated alkyl, unless otherwise stated. Alkyl groups described herein are generally monovalent, but may also be divalent which may also be described herein as “alkylene” or “alkylenyl” groups. Alkyl can include any number of carbons, such as C₁₋₂, C₁₋₃, C₁₄, C₁₋₅, C₁₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The alkoxy component is as defined above. Examples of the alkoxyalkyl group include, but are not limited to, 2-ethoxy-ethyl and methoxymethyl.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Alkylhydroxy” and “hydroxyalkyl” refers to an alkyl group, as defined above, where at least one of the hydrogen atoms is replaced with a hydroxy group. As for the alkyl group, alkylhydroxy groups can have any suitable number of carbon atoms, such as C₁₋₆. Exemplary alkylhydroxy groups include, but are not limited to, hydroxy-methyl, hydroxyethyl (where the hydroxy is in the 1-or 2-position), hydroxypropyl (where the hydroxy is in the 1-, 2- or 3-position), hydroxy butyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Alkyl amine” and “aminoalkyl” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, methyl amine, dimethyl amine, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. The amino nitrogen can be substituted with 0, 1 or 2 alkyl groups. For example, the aminoalkyl can be di-(C₁-C₆ alkyl)amino, which includes, but is not limited to, —N(CH₃)₂, —N(CH₂CH₃)₂, —N(CH₃)(CH₂CH₃), and —N(CH₂CH₂CH₃)₂. One of skill in the art will appreciate that other alkyl amines are useful in the present invention.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. In some embodiments, cycloalkyls are spirocyclic or bridged compounds. In some embodiments, cycloalkyls are optionally fused with an aromatic ring, and the point of attachment is at a carbon that is not an aromatic ring carbon atom. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3-and 1,4-isomers), cycloheptene, cycloheptadiene, cyclooctene, cyclooctadiene (1,3-, 1,4-and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Cycloalkyl groups can contain one or more double bonds in the ring.

“Heterocycloalkyl” refers to a saturated ring system having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. In some embodiments, heterocycloalkyls are spirocyclic or bridged compounds. In some embodiments, heterocycloalkyls are optionally fused with an aromatic ring, and the point of attachment is at a carbon or heteroatom (e.g., nitrogen atom) that is not an aromatic ring carbon atom. The heterocycloalkyl group can include groups such as aziridine, azetidine, pyrrolidine, piperidine, azepane, azocane, quinuclidine, pyrazolidine, imidazolidine, piperazine (1,2-, 1,3-and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperavinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo [4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridavinyl, triavinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{y}—OR^{x}, —R^{y}—OC(O)—Rₓ, —R_{y}—OC(O)—OR^{x}, —R^{y}—OC(O)—N(R^{x})₂, —R^{y}—N(R^{x})₂, —R^{y}—C(O)R^{x}, —R^{y}—C(O)OR^{x}, —R^{y}—C(O)N(R^{x})₂, —R—Op—R^{z}—C(O)N(R^{x})₂, —R^{y}—N(R^{x})C(O)OR^{x}, —R^{y}—N(R^{x})C(O)R^{x}, —R^{y}—N(R^{x})S(O)ₜR^{x} (where t is 1 or 2), —R^{y}—S(O)ₜR^{x} (where t is 1 or 2), —R^{y}—S(O)ₜOR^{x} (where t is 1 or 2) and —R^{y}—S(O)ₜN(R^{x})₂ (where t is 1 or 2), where each R^{x} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl). cycloalkylalkyl (optionally substituted with halogen, hydroxy, methoxy, or trifluoromethyl). aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{y} is independently a direct bond or a straight or branched alkylene or alkenylene chain, and R^{z} is a straight or branched alkylene or alkenylene chain, and where each of the above substituents is unsubstituted unless otherwise indicated.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4-and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2-and 3-pyrrole, pyridine includes 2-, 3-and 4-pyridine, imidazole includes 1-, 2-, 4-and 5-imidazole, pyrazole includes 1-, 3-, 4-and 5-pyrazole, triazole includes 1-, 4-and 5-triazole, tetrazole includes 1-and 5-tetrazole, pyrimidine includes 2-, 4-, 5-and 6-pyrimidine, pyridazine includes 3-and 4-pyridazine, 1,2,3-triazine includes 4-and 5-triazine, 1,2,4-triazine includes 3-, 5-and 6-triazine, 1,3,5-triazine includes 2-triazine, thiophene includes 2-and 3-thiophene, furan includes 2-and 3-furan, thiazole includes 2-, 4-and 5-thiazole, isothiazole includes 3-, 4-and 5-isothiazole, oxazole includes 2-, 4-and 5-oxazole, isoxazole includes 3-, 4-and 5-isoxazole, indole includes 1-, 2-and 3-indole, isoindole includes 1-and 2-isoindole, quinoline includes 2-, 3-and 4-quinoline, isoquinoline includes 1-, 3-and 4-isoquinoline, quinazoline includes 2-and 4-quinoazoline, cinnoline includes 3-and 4-cinnoline, benzothiophene includes 2-and 3-benzothiophene, and benzofuran includes 2-and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4-and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4-and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4-and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4-and 1,3,5-isomers), thiazole, isothiazole, oxazole, isoxazole, quinoxaline, quinazoline, phthalazine, and cinnoline.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992): Lloyd, The Art, Science and Technology of Pharmaceutical Compounding (1999): Pickar, Dosage Calculations (1999): and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Treat”, “treating” and “treatment” refers to any indicia of success in the treatment or amelioration of an injury, pathology, condition, or symptom (e.g., pain), including any objective or subjective parameter such as abatement: remission: diminishing of symptoms or making the symptom, injury, pathology or condition more tolerable to the patient: decreasing the frequency or duration of the symptom or condition: or, in some situations, preventing the onset of the symptom. The treatment or amelioration of symptoms can be based on any objective or subjective parameter: including, e.g., the result of a physical examination.

“Disease” refers abnormal cellular function in an organism, which is not due to a direct result of a physical or external injury. Diseases can refer to any condition that causes distress, dysfunction, disabilities, disorders, infections, pain, or even death. Diseases include, but are not limited to hearing loss.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” or “living organism” refers to animals such as mammals, including, but not limited to, primates (e.g., humans), cows, sheep, goats, horses, dogs, cats, rabbits, rats, mice and the like. In some embodiments, the subject is a human.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A} or 5HT_{2C}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 μM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist. “Functionally selective agonist” refers to a modulator that produces one or a subset of biological responses that are possible from activation of a receptor. For example, activation of 5HT_{2A} receptors is known to cause many downstream effects including increased neural plasticity, increased intracellular calcium concentrations, and hallucinations, among many other biological responses. A functionally selective agonist would produce only a subset of the biological responses possible from activation of the 5HT_{2A} receptor.

“Antagonism” refers to the inactivation of a receptor or enzyme by a modulator, or antagonist. Antagonism of a receptor, for example, is when a molecule binds to the receptor and does not allow activity to occur. “Functionally selective antagonists” block one signaling pathway while leaving others in tact.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

### III. Compounds

The present invention provides tetracyclic heterocyclic compounds useful for the treatment of a variety of diseases and disorders including hearing loss.

In some embodiments, the present invention provides a compound, or a pharmaceutically acceptable salt thereof, having a structure of Formula (J):

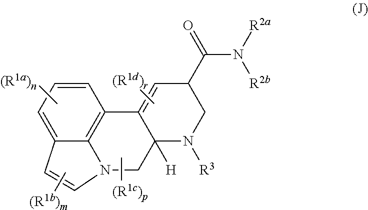

wherein: each R^{1a}, R^{1b}, R^{1c}, and R^{1d} is independently H, C₁₋₆ alkyl, C₂₋₆ alkenyl, C₂₋₆ alkynyl, C₁₋₆ alkoxy, C₁₋₆ alkoxyalkyl, halogen, C₁₋₆ haloalkyl, C₁₋₆ haloalkoxy, —NO₂, or —CN; alternatively, two R^{1a} groups on adjacent ring atoms are combined to form a C₄₋₈ cycloalkyl or 4 to 8 membered heterocycloalkyl having 1 to 2 heteroatoms, each independently N, O, or S; R^{2a} and R^{2b} are each independently H, C₁₋₆ alkyl, C₃₋₆ cycloalkyl, C₁₋₆ alkoxy, C₁₋₆ alkoxyalkyl, C₁₋₆ haloalkyl, or C₁₋₆ haloalkoxy; alternatively, R^{2a} and R^{2b} are combined to form a 4 to 8 membered heterocycloalkyl having 1 to 2 heteroatoms, each independently N, O, or S; R³ is H, C₁₋₆ alkyl, C₁₋₆ alkoxy, C₁₋₆ alkoxyalkyl, C₁₋₆ haloalkyl, or C₁₋₆ haloalkoxy; subscripts m and p are each independently 0 to 2; and subscripts n and r are each independently 0 to 3.

In some embodiments, provided herein is a compound, or a pharmaceutically acceptable salt thereof, having a structure of Formula (I):

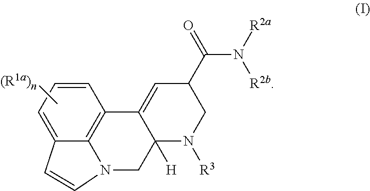

In some embodiments, provided herein is a compound, or a pharmaceutically acceptable salt thereof, having a structure of Formula (Ia), Formula (Ib), Formula (Ic), or Formula (Id):

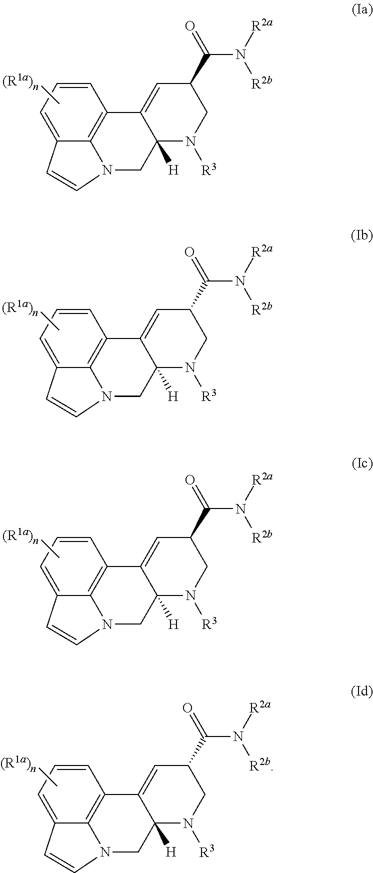

In some embodiments. provided herein is a compound having a structure of Formula (Ia):

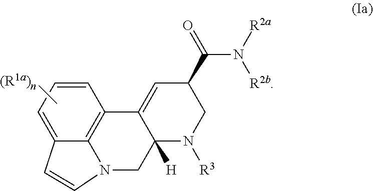

In some embodiments. provided herein is a compound having a structure of Formula (Ib):

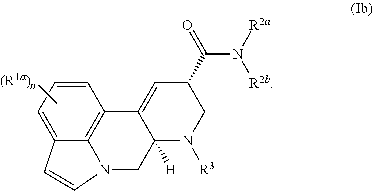

In some embodiments, provided herein is a compound having a structure of Formula (Ic):

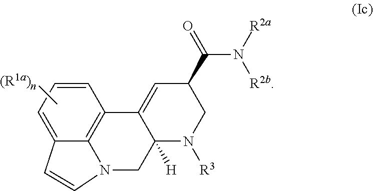

In some embodiments, provided herein is a compound having a structure of Formula (Id):

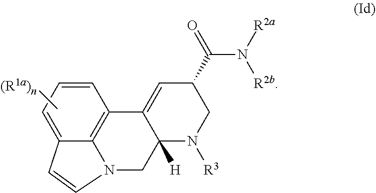

R^{1a} can be any suitable functional group. In some embodiments, R1^{1a} is H, C₁₋₆ alkyl, C₂₋₆ alkenyl, C₂₋₆ alkynyl, C₁₋₆ alkoxy, C₁₋₆ alkoxyalkyl, halogen, C₁₋₆ haloalkyl, C₁₋₆ haloalkoxy, —NO₂, or —CN. In some embodiments, R^{1a} is H, C₁₋₆ alkyl, C₁₋₆ alkoxy, or halogen. In some embodiments, R^{1a} is H, C₁₋₆ alkoxy, or halogen. In some embodiments, R^{1a} is H.

R^{2a} and R^{2b} can be any suitable functional group. In some embodiments, R^{2a} and R^{2b} are each independently H, C₁₋₆ alkyl, C₃₋₆ cycloalkyl, C₁₋₆ alkoxy, C₁₋₆ alkoxyalkyl, C₁₋₆ haloalkyl, or C₁₋₆ haloalkoxy; alternatively, R^{2a} and R^{2b} are combined to form a 4 to 8 membered heterocycloalkyl having 1 to 2 heteroatoms, each independently N, O, or S. In some embodiments, R^{2a} and R^{2b} are each independently H, C₁₋₆ alkyl, C₃₋₆ cycloalkyl, C₁₋₆ alkoxyalkyl, or C₁₋₆ haloalkyl. In some embodiments, R^{2a} and R^{2b} are each independently H or C₁₋₆ alkyl. In some embodiments, R^{2a} and R^{2b} are each independently C₁₋₆ alkyl. In some embodiments, R^{2a} and R^{2b} are each independently ethyl.

R³ can be any suitable functional group. In some embodiments, R³ is H, C₁₋₆ alkyl, C₁₋₆ alkoxy, C₁₋₆ alkoxyalkyl, C₁₋₆ haloalkyl, or C₁₋₆ haloalkoxy. In some embodiments, R³ is H or C₁₋₆ alkyl. In some embodiments, R³ is C₁₋₆ alkyl. In some embodiments, R³ is methyl.

Subscripts m, n, p, and r can be any suitable integer. In some embodiments, subscripts m and p are each independently 0 to 2; and subscripts n and r are each independently 0 to 3. In some embodiments, n is 0.

In some embodiments, provided herein is a compound or a pharmaceutically acceptable salt thereof, having the following structure:

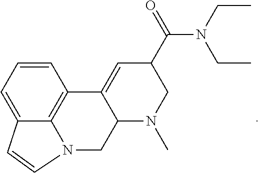

In some embodiments, provided herein is a compound or a pharmaceutically acceptable salt thereof, having the following structure:

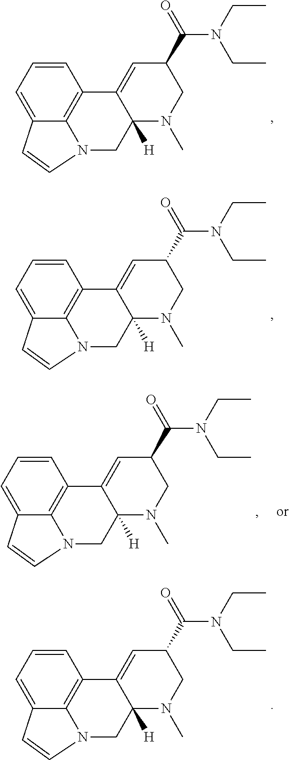

In some embodiments, provided herein is a compound or a pharmaceutically acceptable salt thereof, having the following structure:

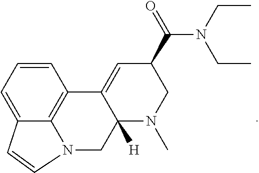

In some embodiments, provided herein is a compound or a pharmaceutically acceptable salt thereof, having the following structure:

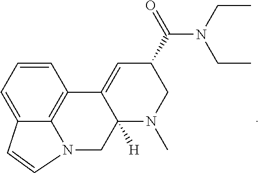

In some embodiments, provided herein is a compound or a pharmaceutically acceptable salt thereof, having the following structure:

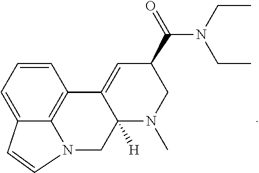

In some embodiments, provided herein is a compound or a pharmaceutically acceptable salt thereof, having the following structure:

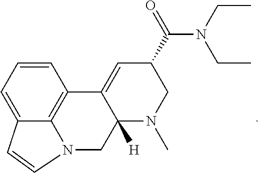

In some embodiments, the compound is a compound of Formula IIa:

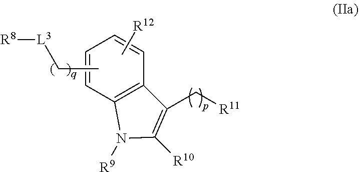

wherein:
L³ is a bond, —C(O)NR^{b}—, —NR^{b}C(O)—, —NHC(O)NR^{b}—, —C(O)O—, —OC(O)—, —NHC(O)O—, —SO₂NR^{b}—, —NHSO₂—, —SO₂—, —O—, —S—, or —NR^{b}—;
R⁸ is hydrogen, halogen, C₁-C₆ alkyl, C₂-C₆ alkenyl, C₃-C₈ cycloalkyl, C₁-C₆ haloalkyl, C₁-C₆ hydroxyalkyl, C₁-C₆ alkoxy, C₁-C₆ aminoalkyl, heterocycloalkyl, aryl, or heteroaryl;
R^{b} is hydrogen, C₁-C₆ alkyl, C₂-C₆ alkenyl, C₃-C₈ cycloalkyl, C₁-C₆ haloalkyl, C₁-C₆ hydroxyalkyl, or C₁-C₆ alkoxy;
R⁹ is hydrogen, C₁-C₆ alkyl, or C₂-C₆ alkenyl:
R¹⁰ is hydrogen, halogen, C₁-C₆ alkyl, C₂-C₆ alkenyl, or C₁-C₆ haloalkyl:
R¹¹ is C₁-C₆ alkylamino, di-(C₁-C₆ alkyl)amino, N-(C₁-C₆ alkyl)pyrrolidinyl, or
   N-(C₁-C₆ alkyl)piperidinyl:
R¹² is hydrogen, halogen, —OH, C₁-C₆ alkyl, C₁-C₆ haloalkyl, C₁-C₆ hydroxyalkyl, or C₁-C₆ alkoxy:
subscript p is an integer from 0 to 3; and
subscript q is an integer from 0 to 3.

In some embodiments, each of R⁹ and R¹⁰ is hydrogen. In some embodiments, the compound is a compound of Formula IIa-1:

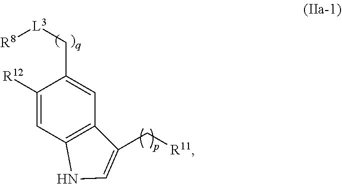

wherein L³, R⁸, R^{b}, R¹¹, R¹², subscripts q, and p are defined previously.

In some embodiments, L³ is a bond, —C(O)NH—, —SO₂NH—, and —SO₂—. In some embodiments, L³ is a bond. In some embodiments, L³ is —C(O)NH—. In some embodiments, L³ is —SO₂NH—. In some embodiments, L³ is —SO₂—.

In some embodiments, R⁸ is hydrogen, C₁-C₆ alkyl, heterocycloalkyl, aryl, or heteroaryl.

In some embodiments, R⁸ can be hydrogen or C₁-C₆ alkyl. In some embodiments, R⁸ is hydrogen. Non-limiting examples of C₁-C₆ alkyl include methyl, ethyl and propyl. In some embodiments, R⁸ is methyl.

In some embodiments, R⁸ can be heterocycloalkyl. Non-limiting examples of heterocycloalkyl include pyrrolidine, piperidine, tetrahydrofuran, tetrahydrothiophene, pyrazolidine, imidazolidine, piperazine, oxazolidine, isoxazolidine, thiazolidine, isothiazolidine, and morpholine. In some embodiments, R⁸ is

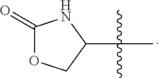 [SMILES: *C1COC(=O)N1]

In some embodiments, R⁸ is 1-pyrrolidinyl. In some embodiments, R⁸ is 1-piperidinyl.

In some embodiments, R⁸ can be aryl. Non-limiting examples of aryl include phenyl, naphthyl and biphenyl. In some embodiments, R⁸ is phenyl.

In some embodiments, R⁸ can be heteroaryl. Non-limiting examples of heteroaryl include pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. In some embodiments, R⁸ is triazolyl. In some embodiments, R⁸ is 1,2,4-triazolyl.

In some embodiments, R¹¹ is di-(C₁-C₆ alkyl)amino, N-(C₁-C₆ alkyl)pyrrolidinyl, or N-(C₁-C₆ alkyl)piperidinyl.

In some embodiments, R¹¹ can be di-(C₁-C₆ alkyl)amino. Non-limiting examples of di-(C₁-C₆ alkyl)amino include —N(CH₃)₂, —N(CH₂CH₃)₂, —N(CH₃)(CH₂CH₃), and —N(CH₂CH₂CH₃)₂. In some embodiments, R¹¹ is —N(CH₃)₂.

In some embodiments, R¹¹ can be N-(C₁-C₆ alkyl)pyrrolidinyl. Non-limiting examples of N-(C₁-C₆ alkyl)pyrrolidinyl include N-methylpyrrolidinyl. In some embodiments, R¹¹ is N-methyl-2-pyrrolidinyl.

In some embodiments, R¹¹ can be N-(C₁-C₆ alkyl)piperidinyl. Non-limiting examples of N-(C₁-C₆ alkyl)piperidinyl include N-methylpiperidinyl. In some embodiments, R¹¹ is N-methyl-4-piperidinyl.

In some embodiments, R¹² is hydrogen, halogen, —OH, C₁-C₆ hydroxyalkyl, or C₁-C₆ alkoxy. In some embodiments, R¹² is hydrogen. R¹² can be halogen including —Cl, Br, or —F. In some embodiments, R¹² is —F. R¹² can be C₁-C₆ alkoxy including methoxy or ethoxy. In some embodiments, R¹² is methoxy.

In some embodiments, subscript p is an integer from 0 to 2. In some embodiments, subscript p is 0. In some embodiments, subscript p is 1. In some embodiments, subscript p is 2.

In some embodiments, subscript q is an integer from 0 to 2. In some embodiments, subscript q is 0. In some embodiments, subscript q is 1. In some embodiments, subscript q is 2.

Other tryptamines useful in the methods of the present invention include, but are not limited to, N,N-dimethyltryptamine, 4-methyl-DMT, 4-methoxy-DMT, 4-hydroxy-N-methyltryptamine, 4-phosphoryloxy-DMT (psilocybin), 5-hydroxy-tryptamine (serotonin), 5-hydroxy-N-methyltryptamine (norbufotenin), 5-hydroxy-DMT (bufotenin), 5-methoxy-DMT, 5-ethoxy-DMT, 5-ethyl-DMT, 5-isopropyl-DMT, 5-t-butyl-DMT, 5-fluoro-DMT, 5-chloro-DMT, 5-iodo-DMT, 5-trifluoromethyl-DMT, 5-nitro-DMT, 5-cyano-DMT, 6-methyl-DMT, 6-hydroxy-DMT, 6-methoxy-DMT, 6-bromo-DMT, 6-fluoro-DMT, 6-chloro-DMT, 5,6-dibromo-tryptamine, 5,6-dibromo-N-methyltryptamine, 5,6-dibromo-DMT, 7-methyl-DMT, 7-ethyl-DMT, 7-chloro-DMT, 7-bromo-DMT, 7-methoxy-DMT, sumatriptan, rizatriptan, zolmitriptan, almotriptan, eletriptan, frovatriptan, and naratriptan.

In some embodiments, the compound of Formula IIa has the structure:

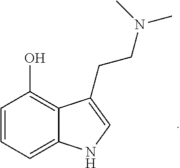

The compounds of the present invention can also be in the salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of the present invention are useful in assays of the tissue distribution of the compounds and their prodrugs and metabolites: preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of this invention can generally be prepared according to the methods known by one of skill in the art by substituting an isotopically-labeled reagent for a non-isotopically labeled reagent. Compounds of the present invention can be isotopically labeled at positions adjacent to the basic amine, in aromatic rings, and the methyl groups of methoxy substituents.

The present invention includes all tautomers and stereoisomers of compounds of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

In some embodiments, a compound provided herein, including pharmaceutically acceptable salts and solvates thereof, is a non-hallucinogenic psychoplastogen.

### IV. Pharmaceutical Compositions and Formulations

In some embodiments, provided herein is a pharmaceutical composition, comprising a therapeutically effective amount of a compound of the present invention, or a pharmaceutically acceptable salt thereof.

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate: magnesium stearate: talc: pectin: dextrin: starch: tragacanth: a low melting wax: cocoa butter; carbohydrates: sugars including, but not limited to, lactose, sucrose, mannitol, or sorbitol, starch from corn, wheat, rice, potato, or other plants: cellulose such as methyl cellulose, hydroxypropylmethyl-cellulose, or sodium carboxymethylcellulose: and gums including arabic and tragacanth: as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin: or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### V. Methods of Treatment

In some embodiments, provided herein is a method of treating a disease or disorder, such as, but not limited to a hearing loss, comprising administering to a subject in need thereof, a therapeutically effective amount of a compound of the present invention, or a pharmaceutically acceptable salt thereof, thereby treating the disease or disorder.

In some embodiments, provided herein is a method of treating a disease, comprising administering to a subject in need thereof, a therapeutically effective amount of a compound of the present invention, or a pharmaceutically acceptable salt thereof, thereby treating the disease.

Hearing loss

In some embodiments, provided herein is a method of treating hearing loss with a compound provided herein (e.g., a compound of Formula (J), Formula (I), Formula (Ia), Formula (Ib), Formula (Ic), Formula (Id), Formula (IIa), Formula (IIa-1), or a pharmaceutically acceptable salt or solvate thereof).

In some embodiments, a compound provided herein, or pharmaceutically acceptable salts thereof, is a non-hallucinogenic psychoplastogens useful for treating one or more diseases or disorders associated with loss of synaptic connectivity and/or plasticity.

Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In some embodiments, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In some embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In some embodiments wherein a mammal's status does improve, the dose of drug being administered is temporarily reduced or temporarily suspended for a certain length of time (i.e., a “drug holiday”). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In some embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In some embodiments, doses employed for adult human treatment are from about 1 mg to about 1000 mg per day. In some embodiments, the desired dose is conveniently presented in a single dose or in divided doses administered simultaneously or at appropriate intervals, for example as two, three, four or more sub-doses per day.

In some embodiments, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In some embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In some embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In some embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In some embodiments, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In some embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers: the age, weight, sex, diet, and medical condition of the subject). Thus, in some instances, the dosage regimen actually employed varies and, in some embodiments, deviates from the dosage regimens set forth herein.

### VI. Examples

### Materials and Methods

All reagents were obtained from commercial sources and reactions were performed using oven-dried glassware (120° C.) under an inert N₂ atmosphere unless otherwise noted. Air-and moisture-sensitive liquids and solutions were transferred via syringe or stainless-steel cannula. Organic solutions were concentrated under reduced pressure (˜5 Torr) by rotary evaporation. Solvents were purified by passage under 12 psi Ar through activated alumina columns. Chromatography was performed using Fisher Chemical™ Silica Gel Sorbent (230-400 Mesh, Grade 60). Compounds purified by chromatography were typically applied to the adsorbent bed using the indicated solvent conditions with a minimum amount of added dichloromethane as needed for solubility. Thin layer chromatography (TLC) was performed on Merck silica gel 60 F254 plates (250 μm). Visualization of the developed chromatogram was accomplished by fluorescence quenching or by staining with aqueous potassium permanganate or Ehrlich's reagent.

Nuclear magnetic resonance (NMR) spectra were acquired on a Bruker 400 operating at 400 and 100 MHz for ¹H and ¹³C, respectively, and are referenced internally according to residual solvent signals. Data for ¹H NMR are recorded as follows: chemical shift (δ, ppm), multiplicity (s, singlet; d, doublet; t, triplet; q, quartet; quint, quintet; m, multiplet), coupling constant (Hz), and integration. Data for ¹³C NMR are reported in terms of chemical shift (δ, ppm). Infrared spectra were recorded using a Thermo Nicolet iS10 Fourier transform infrared (FT-IR) spectrometer with a Smart iTX Accessory [diamond attenuated total reflection (ATR)] and are reported in the frequency of absorption (ν, cm^{—1}). Liquid chromatography-mass spectrometry (LC-MS) was performed using a Waters LC-MS with an ACQUITY Arc QDa detector.

### General Synthetic Scheme:

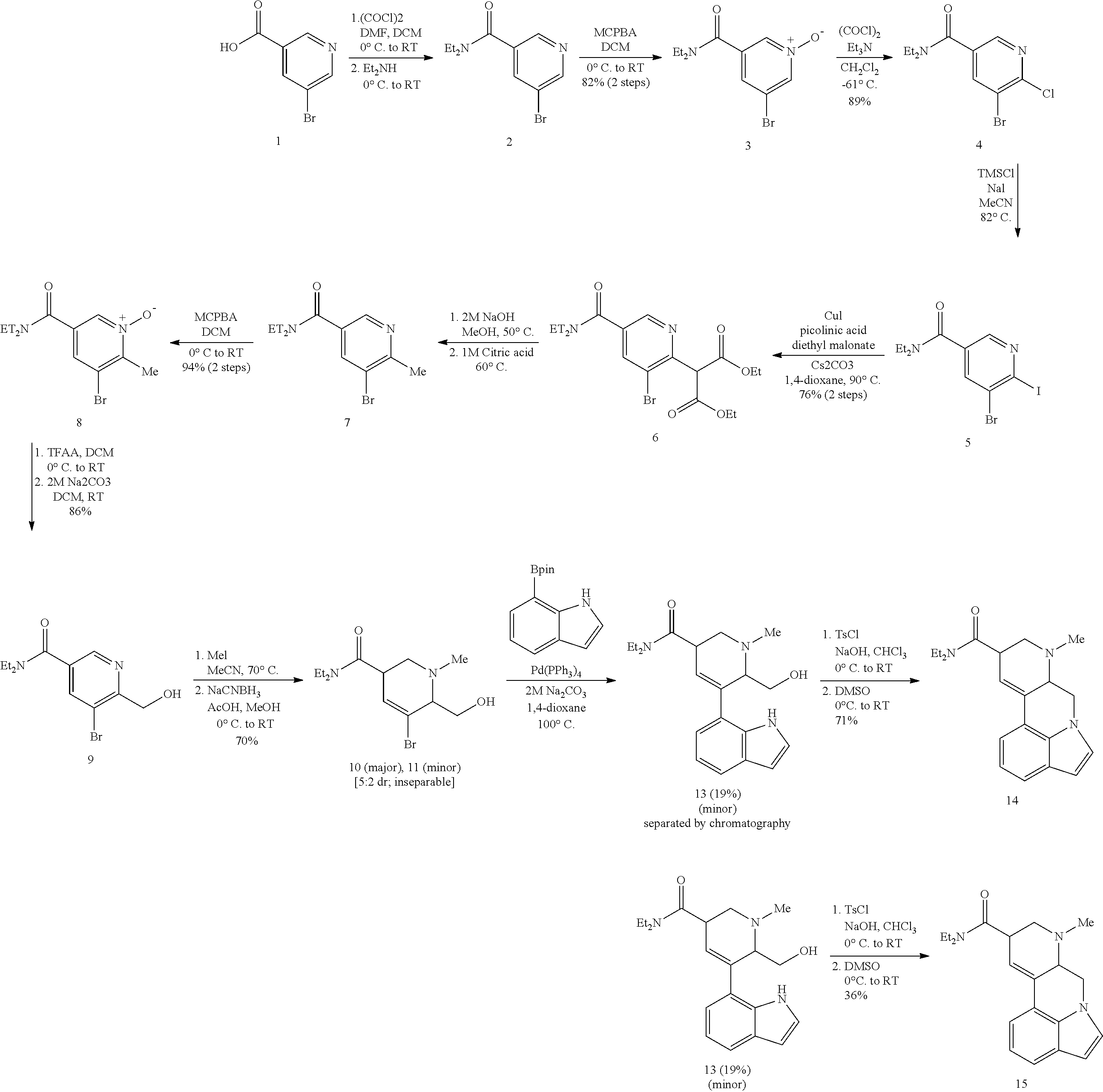

### Example 1

### Preparation of N,N-diethyl-8-methyl-7a,8,9,10-tetrahydro-7H-indolo[7,1-fg][1,7]naphthyridine-10-carboxamide (14, Major Diastereomer)

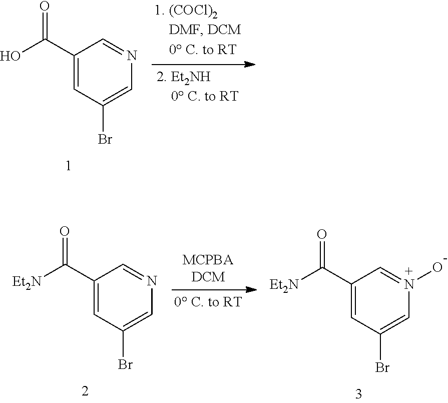

To a 0° C. cooled mixture of 5-bromonicotinic acid (10.000 g, 49.503 mmol, 1.0 equiv) in DCM (250 mL) was added oxalyl chloride (6.37 mL, 74.2 mmol, 1.5 equiv) slowly. To the suspension was added DMF (0.5 mL) dropwise, and the mixture was warmed to ambient temperature and stirred for 1 h. The mixture was cooled to 0° C. and a solution of diethylamine (25.61 mL, 247.5 mmol, 5.0 equiv) in DCM (250 mL) was added slowly via cannula. The mixture was warmed to ambient temperature and stirred for 30 min. H₂O (500 mL) was added, followed by 2M HCl (40 mL) until the pH=1 to 2. The layers were separated, and the aqueous layer was further extracted with DCM (3×200 mL). The organic extracts were combined and sequentially washed with saturated aqueous NaHCO₃ (1×250 mL) and brine (1×250 mL). The organic extract was dried over Na₂SO₄, and concentrated under reduced pressure.

To a 0° C. cooled solution of the resulting brown oil in DCM (200 mL) was added MCPBA (70-75% balance) (22.781 g, 99.006 mmol, 2.0 equiv). The mixture was warmed to ambient temperature and stirred for 18 h. To the solution was added saturated aqueous NaHCO₃ (500 mL) and then 1M NaOH (500 mL). The layers were separated, and the aqueous layer was further extracted with 10% IPA in DCM (3×200 mL). The organic layers were combined, dried over Na₂SO₄, filtered, and concentrated under reduced pressure. The residue was purified via chromatography on silica gel (EtOAc then 12% MeOH in EtOAc) and concentrated under reduced pressure. The resulting pale yellow oil was dissolved in DCM (50 mL), and to the solution was added hexanes (500 mL) slowly with vigorous stirring. The suspension was cooled to 0° C., filtered, and washed with 100 mL cold hexanes to afford 3 (11.041 g, 82%) as a white solid.

¹H NMR (400 MHZ, CDCl₃) δ=8.32 (t, J=1.5 Hz, 1H), 8.09 (t, J=1.3 Hz, 1H), 7.35 (t, J=1.3 Hz, 1H), 3.54-3.46 (m, 2H), 3.29-3.21 (m, 2H), 1.24-1.12 (m, 6H) ppm. ¹³C NMR (100 MHZ, CDCl₃) δ=164.3, 141.0, 136.3, 135.9, 126.4, 120.7, 43.5, 39.9, 14.4, 12.8 ppm. LRMS (ES⁺) m/z [M+H]⁺ calcd for C₁₀H₁₄BrN₂O₂⁺ 273.02; Found 273.12. IR (diamond, ATR) ν 3445, 3068, 2973, 2934, 1633 cm⁻¹.

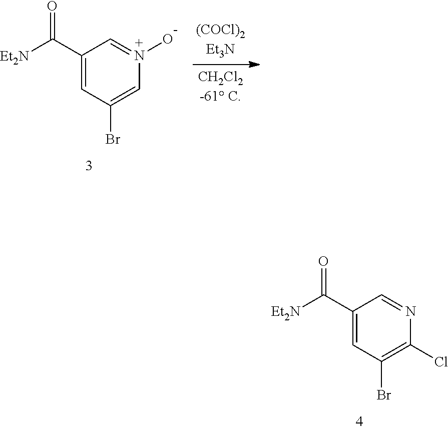

To a −61° C. cooled (CHCl₃/dry ice) solution of 3 (9.900 g, 36.395 mmol, 1.0 equiv) and Et₃N (10.15 mL, 72.79 mmol, 2.0 equiv) in DCM (180 mL) was added oxalyl chloride (6.24 mL, 72.8 mmol, 2.0 equiv) slowly dropwise. The mixture was stirred for 30 minutes, then MeOH (5 mL) was added slowly before warming to ambient temperature, then saturated aqueous NaHCO₃ (25 mL) was added. The solution was poured into 1M NaOH (600 mL) and the layers were separated. The aqueous layer was further extracted with DCM (3×150 mL). The organic extracts were combined, washed with brine (250 mL), dried over Na₂SO₄, and concentrated under reduced pressure. The residue was purified by chromatography on silica gel (25% EtOAc in hexanes) to afford 4 (9.442 g, 89%) as a crystalline white solid.

¹H NMR (400 MHZ, CDCl₃) δ=8.34 (s, 1H), 7.96 (s, 1H), 3.59-3.42 (m, 2H), 3.36-3.18 (m, 2H), 1.28-1.10 (m, 6H) ppm. ¹³C NMR (100 MHZ, CDCl₃) δ=166.0, 151.5, 145.3, 140.7, 133.0, 120.5, 43.6, 39.9, 14.4, 12.9 ppm. LRMS (ES⁺) m/z [M+H]⁺ calcd for C₁₀H₁₃BrClN₂O⁺ 290.99; Found 291.00. IR (diamond, ATR) ν 2974, 2935, 1627, 1574 cm⁻¹.

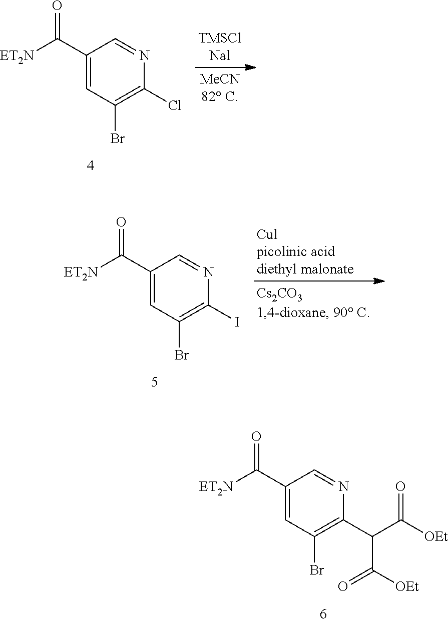

To a vigorously stirred mixture of 4 (5.000 g, 17.243 mmol, 1.0 equiv) and NaI (20.676 g, 137.94 mmol, 8.0 equiv) in acetonitrile (40 mL) was added TMSCl (3.28 mL, 25.86 mmol, 1.5 equiv) slowly. The mixture was stirred at ambient temperature for 30 min, then heated at reflux for 1 hour, with ¼ of the reaction volume removed and collected in a Dean-Stark receiver during this time period. The resulting yellow suspension was cooled to ambient temperature, diluted with DCM (150 mL), and added to a saturated aqueous NaHCO₃ solution (250 mL). With vigorous stirring, a saturated aqueous Na₂S₂O₈ solution (100 mL) was added, followed by 1M NaOH (80 mL). The resultant clear solution was transferred to a separatory funnel and the layers were separated. The aqueous layer was further extracted with DCM (3×100 mL). The organic extracts were combined, washed with brine (200 mL), dried over Na₂SO₄, and concentrated under reduced pressure.

The resulting pale orange solid was added to a sealable screw cap flask along with copper (I) iodide (0.164 g, 0.861 mmol. 0.05 equiv), picolinic acid (0.212 g, 1.72 mmol, 0.1 equiv), and Cs₂CO₃ (16.854 g, 51.729 mmol, 3.0 equiv). 1,4-dioxane (43 mL) and diethyl malonate (5.26 mL, 34.5 mmol, 2.0 equiv) were added, and the flask capped. The mixture was stirred and heated at 90° C. for 16 h. The mixture was cooled to ambient temperature and filtered over celite, and the filter cake was washed with EtOAc (200 mL). The filtrate was added to H₂O (500 mL), then 1M HCl (10 mL) was added and the layers were separated. The aqueous layer was further extracted with EtOAc (2×200 mL). The organic extracts were combined, washed with brine (250 mL), dried over Na₂SO₄, and concentrated under reduced pressure. The residue was purified by chromatography on silica gel (20% EtOAc in hexanes to 50% EtOAc in hexanes) to afford 6 (5.461 g, 76%) as a pale yellow oil.

¹H NMR (400 MHZ, CDCl₃) δ=8.52 (d, J=1.8 Hz, 1H), 7.92 (d, J=1.8 Hz, 1H), 5.22 (s, 1H), 4.34-4.23 (m, 4H), 3.62-3.44 (m, 2H), 3.38-3.19 (m, 2H), 1.32-1.10 (m, 12H) ppm. ¹³C NMR (100 MHZ, CDCl₃) δ=166.62, 166.58, 152.5, 142.2, 138.9, 133.5, 121.9, 62.3, 59.9, 43.6, 39.8, 14.5, 14.1, 12.9 ppm. LRMS (ES⁺) m/z [M+H]⁺ calcd for C₁₇H₂₄BrN₂O₅⁺ 415.09; Found 415.19. IR (diamond, ATR) ν 2980, 2937, 1735, 1631 cm⁻¹.

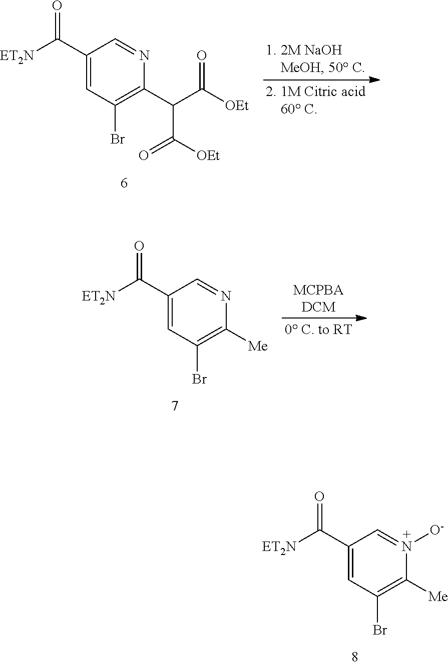

To a solution of 6 (5.350 g, 12.92 mmol, 1.0 equiv) in MeOH (130 mL) was added 2M aq. NaOH (32 mL), and the solution was stirred and heated at 50° C. for 16 h. To the resulting suspension, 1M aq. Citric acid (45 mL) was added to adjust the pH to 4, and the solution was stirred and heated at 60° C. for 24 h. The solution was cooled to ambient temperature and the MeOH was removed by concentration under reduced pressure. The solution was added to H₂O (250 mL) and extracted with DCM (3×200 mL). The organic layers were combined, washed with brine (250 mL), dried over Na₂SO₄, and concentrated under reduced pressure.

To a 0° C. cooled solution of the resulting residue in DCM (50 mL) was added MCPBA (70-75% balance) (5.946 g, 25.84 mmol, 2.0 equiv) slowly. The solution was warmed to ambient temperature and stirred for 22 h. The solution was added to 150 mL 1M NaOH and the layers were separated. The aqueous layer was further extracted with 10% isopropyl alcohol in DCM (3×100 mL). The organic extracts were combined, dried over Na₂SO₄, filtered, and concentrated under reduced pressure. The residue was purified by column chromatography on silica gel (EtOAc then 10% MeOH in EtOAc) to afford 8 (3.469 g, 94%) as a white solid.

¹H NMR (400 MHZ, CDCl₃) δ=8.19 (d, J=0.9 Hz, 1H), 7.4 (d, J=0.9 Hz, 1H), 3.58-3.39 (m, 2H), 3.36-3.18 (m, 2H), 2.66 (s, 3H), 1.24-1.10 (m, 6H) ppm. ¹³C NMR (100 MHz, CDCl₃) δ=164.6, 150.2, 136.2, 133.0, 127.0, 122.1, 43.5, 39.9, 17.4, 14.4, 12.8 ppm. LRMS (ES⁺) m/z [M+H]⁺ calcd for C₁₁H₁₆BrN₂O₂⁺ 287.04; Found 287.12. IR (diamond, ATR) ν 3455, 2972, 2935, 1632 cm⁻¹.

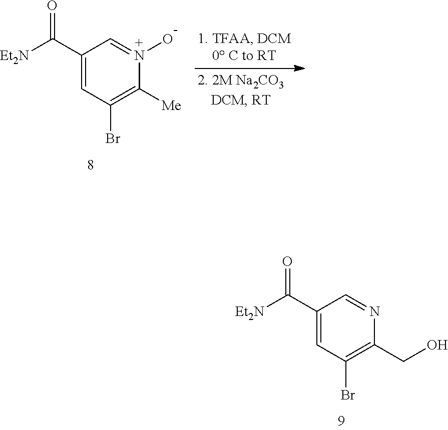

To a 0° C. cooled solution of 8 (1.301 g, 4.531 mmol, 1.0 equiv) in DCM (22.6 mL) was added trifluoroacetic anhydride (1.57 mL, 11.3 mmol, 2.5 equiv) dropwise. The solution was warmed to ambient temperature and stirred for 4 h before concentrating under reduced pressure. The residue was re-dissolved in DCM (22.6 mL) and 2M aq. Na₂CO₃ (45.2 mL) was added. The biphasic solution was stirred vigorously at ambient temperature for 18 h, then poured into H₂O (100 mL). The layers were separated and the aqueous layer was further extracted with DCM (3×50 mL). The organic extracts were combined, washed with brine (100 mL), dried over Na₂SO₄, and concentrated under reduced pressure. The residue was purified by column chromatography on silica gel (EtOAc) to afford 9 (1.119 g, 86%) as a yellow oil.

¹H NMR (400 MHZ, CDCl₃) δ=8.53 (d, J=1.7 Hz, 1H), 7.90 (d, J=1.7 Hz, 1H), 4.77 (d, J=4.7 Hz, 2H), 4.23 (t, J=4.7 Hz, 1H), 3.64-3.46 (m, 2H), 3.38-3.18 (m, 2H), 1.32-1.08 (m, 6H) ppm. ¹³C NMR (100 MHZ, CDCl₃) δ=166.8, 157.6, 144.1, 138.6, 133.2, 118.7, 63.4, 43.6, 39.9, 14.5, 12.9 ppm. LRMS (ES⁺) m/z [M+H]⁺ calcd for C₁₁H₁₆BrN2O₂ 287.04; Found 287.12. IR (diamond, ATR) ν 3412, 2972, 2934, 1624, 1588 cm⁻¹.

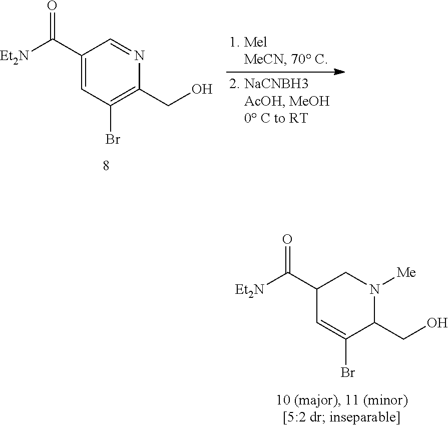

To a solution of 9 (0.980 g, 3.413 mmol, 1.0 equiv) in MeCN (4.25 mL) in a vial was added MeI (1.28 mL, 20.5 mmol, 6.0 equiv). The vial was capped and the solution was heated with stirring at 70° C. for 24 h then subsequently cooled to ambient temperature. To the mixture was added EtOAc (8.5 mL) followed by hexanes (8.5 mL) with vigorous stirring. The suspension was cooled to 0° C., filtered, and washed with hexanes (2×5 mL). The resulting yellow solid was dried under reduced pressure and used directly in the next step.

To a 0° C. cooled solution of the resulting methyl pyridinium salt (1.285 g, 2.995 mmol, 1.0 equiv) in MeOH (30 mL) was added AcOH (0.51 mL, 8.9 mmol, 3.0 equiv) followed by the dropwise addition of NaCNBH₃ (0.565 g, 8.98 mmol, 3.0 equiv) in MeOH (6 mL). The solution was warmed to ambient temperature and stirred for 16 h, then concentrated under reduced pressure. The residue was dissolved in EtOAc (100 mL) and added to 1M NaOH (200 mL). The layers were separated, and the aqueous layer was further extracted with EtOAc (3×100 mL). The organic extracts were combined and washed with brine (150 mL), dried over Na₂SO₄, and concentrated under reduced pressure. The residue was purified by column chromatography on silica gel (3% MeOH in DCM) to afford an inseparable mixture of diastereomers 10 (major diastereomer) and 11 (minor diastereomer) (0.726 g, 70%), 5:2 dr) as a pale yellow oil.

¹H NMR (400 MHZ, CDCl₃) δ=6.21^{†} (s, 1H), 6.11* (d, J=2.8 Hz, 0.4H), 3.94^{†} (dd, J=2.0 Hz, 1H), 3.88-3.78 (m, 1.4H), 3.70-3.64* (m, 0.4H), 3.59* (dd, J=8.8, 11.6 Hz, 0.4H), 3.54-3.47^{†} (m, 1H), 3.34 (quint, J=7.3 Hz, 5.6H), 3.21* (dd, J=9.4, 13.6 Hz, 0.4H), 3.14-3.08* (m, 0.4H), 3.02-2.92^{†} (m, 2H), 2.88-2.80^{†} (m, 1H), 2.76* (dd, J=5.1, 14 Hz, 0.4H), 2.55* (s, 1.2H), 2.45^{†} (s, 3H), 2.24-2.14 (m, 4.2H), 1.09 (t, J=7.1 Hz, 4.2H) ppm. ¹³C NMR (100 MHZ, CDCl₃) δ=170.22, 170.15, 130.4, 127.6, 123.3, 122.9, 68.6, 67.6, 60.9, 59.6, 53.6, 47.0, 43.5, 42.9, 42.2, 42.1, 41.3, 40.7, 40.4, 36.7, 15.1, 14.9, 13.2, 13.1 ppm. LRMS (ES⁺) m/z [M+H]⁺ calcd for C₁₂H₂₂BrN₂O₂⁺305.09; Found 305.14. IR (diamond, ATR) ν 3418, 2970, 2934, 2799, 1629 cm⁻¹.

^{†}denotes ¹H NMR signal arising exclusively from the major diastereomer; *denotes ¹H NMR signal arising exclusively from the minor diastereomer; undesignated signals arise from a mixture of both.

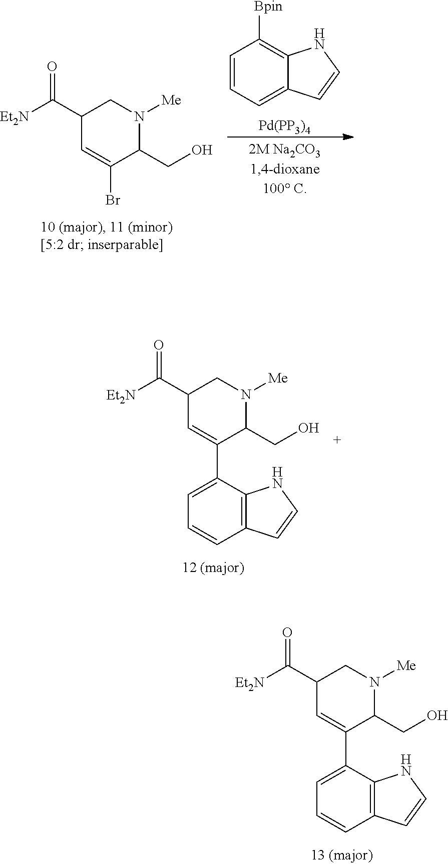

A mixture of diastereomers 10 and 11 (5:2 dr) (0.698 g, 2.29 mmol, 1.0 equiv), 1,4-dioxane (22.9 mL), indole-7-boronic acid pinacol ester (0.834 g, 3.43 mmol, 1.5 equiv), and 2M aq. Na₂CO₃ (2.29 mL) were added to a vial and the solution was sparged with N₂ for 10 min before the addition of Pd(PPh₃)₄ (0.132 g, 0.114 mmol, 0.05 equiv). The vial was capped and the mixture was heated with stirring at 100° C. in a preheated oil bath for 4 h. The mixture was cooled to ambient temperature, added to H₂O (400 mL), and extracted with EtOAc (3×150 mL). The organic extracts were combined, washed with brine (200 mL), dried over Na₂SO₄, and concentrated under reduced pressure. The residue was purified by column chromatography on silica gel (2% MeOH in DCM to 10% MeOH in DCM) to afford 12 (major diastereomer) (0.398 g, 51%) and 13 (minor diastereomer) (0.148 g, 19%) as off white semi-solids.

Major Diastereomer, 12. ¹H NMR (400 MHZ, CDCl₃) δ=9.46 (s, 1H), 7.56 (d, J=7.8 Hz, 1H), 7.22 (t, J=2.8 Hz, 1H), 7.06 (t, J=7.3 Hz, 1H), 7.00 (dd, J=0.9, 8.4 Hz, 1H), 6.53 (dd, J=2.1, 3.2 Hz, 1H), 5.92 (s, 1H), 3.79 (dd, J=3.0, 11.2 Hz, 1H), 3.74-3.66(m, 1H), 3.50-3.28 (m, 5H), 3.26-3.08 (m, 3H), 3.07-2.96 (m, 1H), 2.52 (s, 2H), 1.23 (t, J=7.1 Hz, 3H), 1.10 (t, J=7.1 Hz, 3H) ppm. ¹³C NMR (100 MHZ, CDCl₃) δ=171.7, 137.3, 135.4, 128.11, 128.05, 124,9, 124.1, 121.4, 119.9, 119.4, 102.4, 66.8, 59.1, 54.0, 43.2, 42.0, 40.3, 39.3, 14.9, 13.1 ppm. LRMS (ES⁺) m/z [M+H]⁺ calcd for C₂₀H₂₈N₃O₂⁺ 342.22; Found 342.32. IR (diamond, ATR) ν 3267, 2970, 2932, 1615 cm⁻¹.

Minor Diastereomer, 13. ¹H NMR (400 MHZ, CDCl₃) δ=9.86 (s, 1H), 7.55 (d, J=7.8 Hz, 1H), 7.29-7.24 (m, 1H), 7.07 (t, J=7.4 Hz, 1H), 6.98 (dd, J=0.6, 7.6 Hz, 1H), 6.51 (dd, J=2.1 Hz, 3.2 Hz, 1H), 6.08-6.04 (m, 1H0, 3.76-3.68 (m, 1H), 3.64-3.28 (m, 8H), 3.15-3.06 (m 1H), 2.99 (dd, J=5.7, 13.2 Hz, 1H), 2.66 (s, ³H), 1.26 (t, J=7.2 Hz, ³H), 1.17 (t, J=7.1 Hz, 3H) ppm. ¹³C NMR (100 MHZ, CDCl₃) δ=173.0, 135.7, 135.0, 128.4, 125.6, 125.4, 123.8, 120.0, 119.4, 119.0, 102.1, 64.8, 60.9, 48.6, 42.7, 42.3, 40.6, 34.8, 15.1, 13.3 ppm. LRMS (ES⁺) m/z [M+H]⁺ calcd for C₂₀H₂₈N₃O₂⁺ 342.22; Found 342.32. IR (diamond, ATR) ν 3270, 2973, 2934, 1613 cm⁻¹.

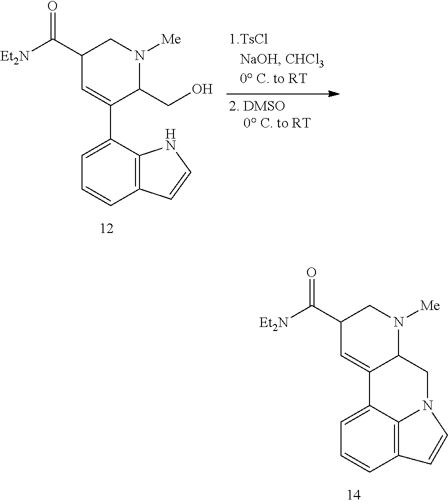

To a 0° C. cooled solution of 12 (0.250 g, 0.732 mmol, 1.0 equiv) in CHCl₃ (7.3 mL) was added freshly crushed NaOH (0.234 g, 5.86 mmol, 8.0 equiv). A solution of TsCl (0.167 g, 0.878 mmol, 1.2 equiv) in CHCl₃ (1.5 mL) was added dropwise over 10 minutes. The mixture was warmed to ambient temperature and stirred for 1.5 h. The mixture was cooled to 0° C., and DMSO (3.7 mL) was added slowly before warming to ambient temperature and stirring for 1 h. The mixture was partitioned in H₂O (250 mL) and EtOAc (200 mL) and the layers were separated. The aqueous layer was further extracted with EtOAc (3×100 mL). The organic extracts were combined, washed with brine (250 mL), dried over Na₂SO₄, and concentrated under reduced pressure. The residue was purified by column chromatography on silica gel (8% MeOH in EtOAc to 12% MeOH in EtOAc) to afford 14 (0.168 g, 71%) as an off white semi-solid.

¹H NMR (400 MHZ, CDCl₃) δ=7.5 (d, J=7.9 Hz, 1H), 7.31 (d, J=7.3 Hz, 1H), 7.08-7.04 (m, 2H), 6.46 (d, J=3.0 Hz, 1H), 6.31 (s, 1H), 4.66 (dd, J=5.4, 11.2 Hz, 1H), 3.90-3.82 (m, 1H), 3.80 (t, J=11.1 Hz, 1H), 3.54-3.40 (m, 5H), 3.05 (dd, J=5.0, 11.2 Hz, 1H), 2.95 (t, J=10.7 Hz, 1H), 2.59 (s, 3H), 1.26 (t, J=7.1Hz, 3H), 1.18 (t, J=7.1Hz, 3H) ppm. ¹³C NMR (100 MHz, CDCl) δ=171.2, 133.2, 132.5, 126.3, 126.2, 120.3, 120.0, 118.91, 118.88, 114.9, 101.3, 60.5, 55.8, 48.0, 44.0, 42.1, 40.3, 39.9, 15.0, 13.2 ppm. LRMS (ES⁺) m/z [M+H]⁺ calcd for C₂₀H₂₆N₃O⁺ 324.21: Found 324.29. IR (diamond, ATR) ν 2972, 2869, 2798, 1636 cm⁻¹.

### Example 2

### Preparation of N,N-diethyl-8-methyl-7a,8,9,10-tetrahydro-7H-indolo[7,1-fg][1,7]naphthyridine-10-carboxamide (15, Minor Diastereomer)

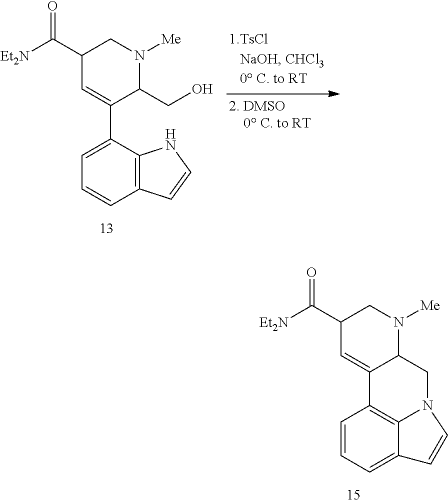

To a 0° C. cooled solution of 13 (0.130 g, 0.381 mmol, 1.0 equiv) in CHCl₃ (3.8 mL) was added freshly crushed NaOH (0.122 g, 3.05 mmol, 8.0 equiv). A solution of TsCl (0.087 g, 0.46 mmol, 1.2 equiv) in CHCl₃ (0.76 mL) was added dropwise over 10 minutes. The mixture was warmed to ambient temperature and stirred for 1.5 h. The mixture was cooled to 0° C., and DMSO (1.9 mL) was added slowly before warming to ambient temperature and stirring for 3 h. The mixture was partitioned in H₂O (200 mL) and EtOAc (150 mL) and the layers were separated. The aqueous layer was further extracted with EtOAc (3×50 mL). The organic extracts were combined, washed with brine (100 mL), dried over Na₂SO₄, and concentrated under reduced pressure. The residue was purified by column chromatography on silica gel 8% MeOH in EtOAc to 12% MeOH in EtOAc) to afford 15 (0.044 g, 36%) as a brown semi-solid.

¹H NMR (400 MHZ, CDCl₃) δ=7.48 (d, J=7.9 Hz, 1H), 7.22 (d, J=7.2 Hz, 1H), 7.08-7.02 (m, 2H), 6.45 (d, J=3.0 Hz, 1H), 6.37 (dd, J=2.0, 3.6 Hz, 1H), 4.50 (dd, J=5.5, 11.2 Hz, 1H), 4.02 (t, J=11.2 Hz, 1H), 3.66-3.60 (m, 1H), 3.56-3.30 (m, 5H), 3.15 (dd, J=5.7, 12.2 Hz, 1H), 2.83 (dd, J=4.8, 12.2 Hz, 1H), 2.62 (s, 3H), 1.27 (t, J=7.0 Hz, 3H), 1.13 (t, J=7.0 Hz, 3H) ppm. ¹³C NMR (100 MHZ, CDCl) δ=171.5, 133.4, 133.4, 126.5, 126.2, 120.4, 120.1, 119.9, 118.9, 114.2, 101.2, 58.6, 52.5, 47.9, 43.6, 42.0, 40.3, 37.4, 15.0, 13.2 ppm. LRMS (ES⁺) m/z [M+H]⁺ calcd for C₂₀H₂₆N₃O⁺ 324.21: Found 324.29. IR (diamond, ATR) ν 2969, 2932, 2871, 2791, 1634 cm⁻¹.

### Example 3

### Biological Assay

Mice were treated with N,N-diethyl-8-methyl-7a,8,9,10-tetrahydro-7H-indolo[7,1-fg][1,7]naphthyridine-10-carboxamide via intraperitoneal injection. After 24 h, the animals were sacrificed by transcardial perfusion of fixative and the inner ear was dissected.

Microscopy methods: Airyscan confocal fluorescence imaging of immunofluorescence stained whole mount organ of Corti.

### Example 4

### Auditory Brainstem Response Assay

Auditory brainstem responses (ABRs) were recorded at baseline (5-6 days prior to treatment) and 24 h post treatment with psilocin (1 mg/kg). Mice were anesthetized via i.p. injections of ketamine (90 mg/kg) and xylazine (20 mg/kg). Subcutaneous electrodes were placed behind the right pinna (inverting) and vertex (active). A ground electrode was placed in the left leg of the mouse. Stimuli were 5-ms tone pips (2 ms cos2 rise-fall) delivered at 21/s with alternating stimulus polarity. Recorded electrical responses were filtered (300 Hz to 3 kHz) and averaged using BioSig software (TDT). The sound intensity level was decreased in 5 dB increments from 90 dB SPL-20 dB SPL. At each sound level 512 responses were averaged.

Wave I amplitudes were measured from the estimated baseline prior to the response to the peak of wave I in μV. ABR wave I amplitudes were analyzed at baseline (5-6 days pre-treatment) and 24 h post-treatment of psilocin (1 mg/kg). Data analyses and statistics were performed using Prism version 9.4 (GraphPad) and presented as mean±SEM. A 2-way ANOVA followed by Sidak's multiple comparison test was performed (n=3). Psilocin treatment after 24 h significantly increased wave I amplitude from baseline at 8 kHz (p<0.01, F(1,14)=11.31) and 16 kHz (p<0.01,F(1,14)=11.94).

### Example 5

### Inner Hair Cell Ribbon Synapse Density 24 h after psilocin Treatment

8-week old C57/B6 mice were injected with 1 mg/kg psilocin or vehicle control (0).9% saline). 24 h later, mice were euthanized then inner ear tissues were collected for immunofluorescence. Isolated cochlea were punched into oval and round windows using a syringe needle and rinsed/fixed with 4% paraformaldehyde in 1×cold PBS. Then, cochlea were further fixed in 4% paraformaldehyde in 1×cold PBS for additional 2 hours at 4C0. Following fixation, samples were rinsed 3×20 min in PBS and dissected under a stereomicroscope to the three turns: apical, middle and basal. Tissues were permeabilized in PBS+0.25% Triton X-100 solution for 10 min at room temperature on a rocking platform; blocked with 10% goat serum and 25 mM glycine for 1 h at RT. Tissues were incubated at 4° C. overnight with the following primary antibodies: monoclonal mouse anti-carboxyl-terminal binding protein 2 (CtBP2) IgG1 at 1:200 (612044: BD Biosciences) counterstained with goat anti-Mouse IgG1 conjugated with Alexa Fluor 568 (#A-21124), monoclonal mouse anti-GluR2 IgG2a at 1:1000 (MAB397: Millipore) counterstained with goat anti-Mouse IgG2a conjugated with Alexa Fluor 488 (#A-21131), and polyclonal rabbit anti-myosin VIIa at 1:200 (NB120-3481; Novus Biologicals) counterstained with goat anti-Rabbit IgG (H+L) conjugated with Alexa Fluor 647 (#A-21244). Antibodies were added with 1% goat serum. The following day, after three 15-min PBS washes, the tissues were incubated with the Alexa-conjugated secondary antibodies at a concentration of 1:600 for 1 h in darkness at room temperature. Following the final washes after secondary incubations, samples were carefully mounted on slides using ProLong Glass antifade media and left to dry for at least 24 h before image acquisition. Frequency regions corresponding to 16 and 8 kHz were located through their distance from cochlear apex, based on the place-frequency map and imaged with a 63×1.4NA Plan Apo objective on a Zeiss 880 LSM Airyscan confocal microscope with a 47 nm xy pixel size and a 110 nm z-step size (Carl Zeiss, Oberkochen, Germany). After acquisition, images were Airyscan processed.

Synapses and hair cells were manually segmented in a blinded fashion using Fiji software, then statistical analyses were performed using Prism (Graphpad) software.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A use of psychoplastogens for treating hearing loss.

2. A use of psychoplastogens for increasing synaptic density in the ear.

3. A use of psychedelics for treating hearing loss.

4. A use of psychedelics for increasing synaptic density in the ear.

5. A use of hallucinogenic psychoplastogens for treating hearing loss.

6. A use of hallucinogenic psychoplastogens for increasing synaptic density in the ear.

7. A use of non-hallucinogenic psychoplastogens for treating hearing loss.

8. A use of non-hallucinogenic psychoplastogens for increasing synaptic density in the ear.

9. A use of psychedelics to protect against noise exposure induced hearing loss.

10. A use of psychedelics to protect against chemical exposure induced hearing loss.

11. A use of psychedelics to protect against aging induced hearing loss.

12. A use of psychoplastogens to protect against noise exposure induced hearing loss.

13. A use of psychoplastogens to protect against chemical exposure induced hearing loss.

14. A use of psychoplastogens to protect against aging induced hearing loss.

15. A use of hallucinogenic psychoplastogens to protect against noise exposure induced hearing loss.

16. A use of hallucinogenic psychoplastogens to protect against chemical exposure induced hearing loss.

17. A use of hallucinogenic psychoplastogens to protect against aging induced hearing loss.

18. A use of non-hallucinogenic psychoplastogens to protect against noise exposure induced hearing loss.

19. A use of non-hallucinogenic psychoplastogens to protect against chemical exposure induced hearing loss.

20. A use of non-hallucinogenic psychoplastogens to protect against aging induced hearing loss.

21. A method of using psychoplastogens to initiate or promote neuritogenesis in a living organism.

22. A method of using psychoplastogens to encourage or promote spinogenesis in a living organism.

23. A method of using hallucinogenic psychoplastogens to initiate or promote neuritogenesis in a living organism.

24. A method of using hallucinogenic psychoplastogens to encourage or promote spinogenesis in a living organism.

25. A method of using non-hallucinogenic psychoplastogens to initiate or promote neuritogenesis in a living organism.

26. A method of using non-hallucinogenic psychoplastogens to encourage or promote spinogenesis in a living organism.

27. A method of using psychoplastogens to initiate or promote neuritogenesis in spiral ganglion neurons.

28. A method of using psychoplastogens to encourage or promote spinogenesis in spiral ganglion neurons.

29. A method of using hallucinogenic psychoplastogens to initiate or promote neuritogenesis in spiral ganglion neurons.

30. A method of using hallucinogenic psychoplastogens to encourage or promote spinogenesis in spiral ganglion neurons.

31. A method of using non-hallucinogenic psychoplastogens to initiate or promote neuritogenesis in spiral ganglion neurons.

32. A method of using non-hallucinogenic psychoplastogens to encourage or promote spinogenesis in spiral ganglion neurons.

33. The use or method of any one of claims 1 to 32, wherein the psychoplastogen is a compound, or a pharmaceutically acceptable salt thereof, having a structure of Formula (J): 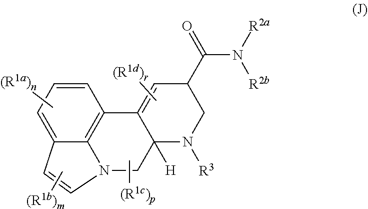
wherein:
each R^{1a}, R^{1b}, R^{1c}, and R^{1d} is independently H, C₁₋₆ alkyl, C₂₋₆ alkenyl, C₂₋₆ alkynyl, C₁₋₆ alkoxy, C₁₋₆ alkoxyalkyl, halogen, C₁₋₆ haloalkyl, C₁₋₆ haloalkoxy, —NO₂, or —CN;
alternatively, two R^{1a} groups on adjacent ring atoms are combined to form a C₄₋₈ cycloalkyl or 4 to 8 membered heterocycloalkyl having 1 to 2 heteroatoms, each independently N, O, or S;
R^{2a} and R^{2b} are each independently H, C₁₋₆ alkyl, C₃₋₆ cycloalkyl, C₁₋₆ alkoxy, C₁₋₆ alkoxyalkyl, C₁₋₆ haloalkyl, or C₁₋₆ haloalkoxy;
alternatively, R^{2a} and R^{2b} are combined to form a 4 to 8 membered heterocycloalkyl having 1 to 2 heteroatoms, each independently N, O, or S;
R³ is H, C₁₋₆ alkyl, C₁₋₆ alkoxy, C₁₋₆ alkoxyalkyl, C₁₋₆ haloalkyl, or C₁₋₆ haloalkoxy;
subscripts m and p are each independently 0 to 2; and
subscripts n and r are each independently 0 to 3.

34. The method or use of any one of claims 1 to 33, or a pharmaceutically acceptable salt thereof, wherein the psychoplastogen has the following structure:

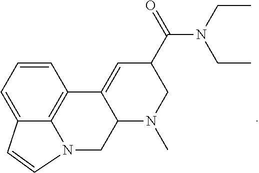

35. The use or method of any one of claims 1 to 32, wherein the psychoplastogen is a compound, or a pharmaceutically acceptable salt thereof, having a structure of Formula (IIa): 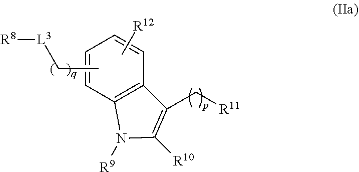
wherein:
L³ is a bond, —C(O)NR^{b}—, —NR^{b}C(O)—, —NHC(O)NR^{b}—, —C(O)O—, —OC(O)—, —NHC(O)O—, —SO₂NR^{b}—, —NHSO₂—, —SO₂—, —O—, —S—, or —NR^{b}—;
R⁸ is hydrogen, halogen, C₁-C₆ alkyl, C₂-C₆ alkenyl, C₃-C₈ cycloalkyl, C₁-C₆ haloalkyl, C₁-C₆ hydroxyalkyl, C₁-C₆ alkoxy, C₁-C₆ aminoalkyl, heterocycloalkyl, aryl, or heteroaryl;
R^{b} is hydrogen, C₁-C₆ alkyl, C₂-C₆ alkenyl, C₃-C₈ cycloalkyl, C₁-C₆ haloalkyl, C₁-C₆ hydroxyalkyl, or C₁-C₆ alkoxy;
R⁹ is hydrogen, C₁-C₆ alkyl, or C₂-C₆ alkenyl;
R¹⁰ is hydrogen, halogen, C₁-C₆ alkyl, C₂-C₆ alkenyl, or C₁-C₆ haloalkyl;
R¹¹ is C₁-C₆ alkylamino, di-(C₁-C₆ alkyl)amino, N-(C₁-C₆ alkyl)pyrrolidinyl, or
N-(C₁-C₆ alkyl)piperidinyl;
R¹² is hydrogen, halogen, —OH, C₁-C₆ alkyl, C₁-C₆ haloalkyl, C₁-C₆ hydroxyalkyl, or C₁-C₆ alkoxy;
subscript p is an integer from 0 to 3; and
subscript q is an integer from 0 to 3.

36. The method or use of any one of claims 1 to 33, or a pharmaceutically acceptable salt thereof. wherein the psychoplastogen has the following structure: [chemical structure]
